(19) United States
(12) Patent Application Publication
(10) Pub. No.: US 2007/0148240 A1
(43) Pub. Date: Jun. 28, 2007

(54) Oral formulation containing itraconazole and methods for manufacturing and using the same

(76) Inventors: Fang-Yu Lee, Taichia (TW); Shan-Chiung Chen, Fong Yuan City (TW); Bin-Ken Chen, Waibu Township (TW); Han-Chiang Kuo, Wan Li Township (TW)

Correspondence Address: ANDREWS KURTH LLP, 1350 I STREET, N.W., SUITE 1100, WASHINGTON, DC, 20005, US

(21) Appl. No.: 11/315,124

(22) Filed: Dec. 23, 2005

(51) Int. Cl. A61K 9/24; A61K 31/496

(52) U.S. Cl. 424/472; 514/254.07

(57) ABSTRACT

The present invention provides oral pharmaceutical formulation for azole antimicrobial drugs such as itraconazole, saperconazole, ketoconazole, and fluconazole. The oral pharmaceutical formulation contains a core and a drug coating layer. The drug coating layer contains the azole antimicrobial drug and a binder, but not containing an emulsion (such as polyoxypropylene-polyoxyethylene block copolymers, polyoxyethylene-sorbitan-fatty acid esters, sodium lauryl sulfate, or vitamin E polyethylene glycol succinate) and/or an absorbent aid (such as DL-malic acid, citric acid, ascorbic acid, and alginic acid). The oral pharmaceutical formulation can optionally contain a protective layer, such as polyethylene glycol 20,000. The present invention also provides a method for preparing and using the formulation.

### FIELD OF THE INVENTION

The present invention relates to an oral pharmaceutical formulation which contains a core and a drug coating layer. The core is preferred to be a round, spherical core which comprises sucrose, lactose, starch, talc, or microcrystalline cellulose or any combination thereof. The preferred drug is an azole antifungal drug, including, but not limited to, itraconazole, saperconazole, ketoconazole, and fluconazole. The drug coating layer includes the drug and a binder, but does not include an emulsion (such as polyoxypropylene-polyoxyethylene block copolymers, polyoxyethylene-sorbitan-fatty acid esters, sodium lauryl sulfate, or vitamin E polyethylene glycol succinate) and an absorbent aid, which is an organic acid (such as DL-malic acid, citric acid, ascorbic acid, and alginic acid). The present invention also relates to a method for making and using the oral pharmaceutical formulation.

### BACKGROUND OF THE INVENTION

U.S. Pat. No. 4,267,179 discloses a number of 1H-imidazole and 1H-1,2,4-triazole derivatives having antifungal and antibacterial properties. Specifically, a number of heterocyclic derivatives of (4-phenyl-1-piperazinyl-aryloxymethyl-1,3-dioxolan-2-yl) methyl-1H-imidazoles and 1H-1,2,4-triazoles are described. Among these azole compounds and their derivatives, itraconazole, saperconazole, ketoconazole, and fluconazole are currently commercially available. These commercially available azole compounds are known for their broad spectrum of antimicrobial activity. For example, they are found to be highly active against a wide variety of fungi such as Microsporum canis, Pityrosporum ovale, Ctenomyces mentagrophytes, Trichophyton rubrum, Phialophora verrucosa, Cryptococcus neoformans, Candida tropicalis, Candida albicans, Mucor species, Aspergillus fumigatus, Sporotricum schenckii and Saprolegnia species. They are also active against bacteria, such as Erysipelotrix insidiosa, Staphylococcus hemolyticus and Streptococcus pyogenes.

Itraconazole is currently commercially available under the trade name Sporanox® in capsule or tablet form from Janssen Pharmaceutica (Beerse, BE). The chemical structure of itraconazole is disclosed in U.S. Pat. No. 4,267,179 as (±)-cis-4-[4-[4-[4-[[2-(2,4-dichlorophenyl)-2-(1H-1,2,4-triazol-1-ylmethyl)-1,3-dioxolan-4-yl]methoxy]phenyl]-1-piperazinyl]phenyl]-2,4-dihydro-2-(1-methylpropyl)-3H-1,2,4-triazol-3-one, having the formula of:

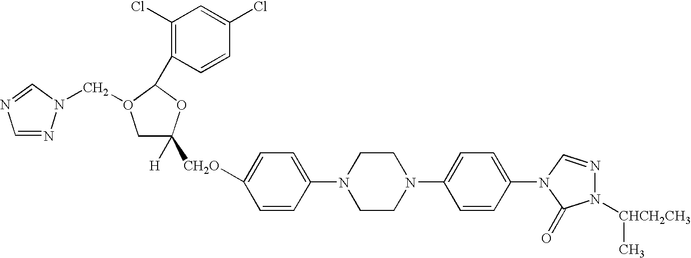

Itraconazole is especially known for its activity against a broad range of fungal inductions such as those caused by Trichophyton rubrum, Tricophyton mentagrophytes, Epidermophyton floccsum and Candida albicans.

The chemical structure of saperconazole is disclosed in U.S. Pat. No. 4,916,134 as (±)-cis-4-[4-[4-[4-[[2-(2,4-difluorophenyl)-2-(1H-1,2,4-triazol-1-ylmethyl)-1,3-dioxolan-4-yl]methoxy]phenyl]-1-piperazinyl]phenyl]-2,4-dihydro-2-(1-methylpropyl)-3H-1,2,4-triazol-3-one. Saperconazole has antimicrobial activity, in particular against fungi belonging to the genus Aspergillus.

Ketoconazole was the first of the azole antifungal agents to become commercially available. The chemical structure of ketoconazole is disclosed in U.S. Pat. No. 4,144,346 as cis-1-acetyl-4-[4-[[2-(2,4-dichlorophenyl)-2-(1H-imidazole-1-ylmethyl)-1,3-dioxolan-4-yl]methoxy]phenyl]piperazine. Ketoconazole is an orally active, broad-spectrum antifungal agent. The compound, an imidazole derivative structurally related to miconazole and clotrimazole, impairs the synthesis of ergosterol, which is the principal sterol of fungal cell membranes.

Fluconazole is a water-soluble triazole with greater than 90% bioavailability after oral administration. The chemical structure of fluconazole is disclosed in U.S. Pat. No. 4,404,216 as 2-(2,4-difluorophenyl)-1,3-bis(1H-1,2,4-triazol-1-yl)propan-2-ol. Fluconazole is used extensively to treat a wide range of Candida infections. In particular, it is widely used in connection with therapy for oropharyngeal candidiasis in patients with advanced HIV infection and AIDS.

The solubility and bioavailability of itraconazole and saperconazole are low due to the fact that these compounds have a low solubility in water and a low pKa value. For example, the solubility of itraconazole is less than 1 μg/ml in water and the pKa value of itraconazole is 3.7. The solubility of itraconazole in ethanol is also low. However, itraconazole is easily soluble in methylene chloride and also easily soluble in acids such as hydrochloric acid, acetic acid, phosphoric acid and methylsulfonic acid.

There have been several reports which show improvement of solubility and bioavailability of itraconzole and/or saperconazole. For example, U.S. Pat. No. 6,100,285 describes a solvent system for dissolving itraconazole. The solvent system contains volatile organic acid solvents such as acetic acid and formic acid, with the solvent itself in an aqueous solution of the acid.

U.S. Pat. No. 5,707,975 discloses a pharmaceutical formulation for itraconazole and saperconazole which is said to have improved solubility and bioavailability. The formulation uses cyclodextrins or the derivatives of cyclodextrins (e.g., hydroxypropyl-β-cyclodextrin) as a solubilizer; an aqueous acidic medium as a bulk liquid carrier (such as hydrochloric acid to achieve optimum pH of 2.0±0.1); and an alcoholic co-solvent (e.g., PEG 400) to dissolve the compounds.

U.S. Pat. No. 5,633,015 (the '015 patent) discloses a pharmaceutical formulation for itraconazole and saperconazole in the form of beads. The beads comprise a central, rounded or spherical core, a coating film, and a seal-coating polymer layer. The core has a diameter of about 600 to about 700 μm (25-30 mesh). The coating film contains a hydrophilic polymer (such as hydroxypropyl methylcellulose) and a drug (e.g., itraconazole and/or saperconazole). The seal-coating polymer layer is applied to the drug coated cores to prevent sticking of the beads, which would have the undesirable effect of a concomitant decrease of the dissolution rate and of bioavailability. The beads use polyethylene glycol (PEG), in particular, PEG 20,000, as the seal-coating polymer.

U.S. Pat. No. 6,039,981 discloses a pharmaceutical composition which comprises a fused mixture of itraconazole and phosphoric acid, a pharmaceutically acceptable carrier, and a surfactant. The fused mixture of itraconazole and phosphoric acid is prepared by heating the mixture to a temperature ranging from 100 to 170° C. to obtain a homogeneous melt mixture.

U.S. Pat. No. 6,485,743 discloses a method and composition of an oral preparation of itraconazole, where itraconazole and hydrophilic polymer (i.e., polyvinylacetal dithylarmoacetate and/or aminoalkyl methacrylate copolymer) are dissolved in solvent, followed by spray-drying prior to dispersions.

U.S. Pat. No. 6,663,897 discloses a method of manufacturing an itraconazole oral dosage form that is substantially free of residual methylene chloride, which requires the addition of a strong acid (preferably an inorganic acid or organic sulphonic acid).

The inventors of the present application recently were granted U.S. Pat. No. 6,673,373 (the '373 patent), which is incorporated herein by reference. The '373 patent discloses an oral antifungal formulation which contains a core, a drug emulsion layer, and a protective layer. The drug emulsion contains an antifungal drug, an emulsion, preferably vitamin E polyethylene glycol succinate, a binder, preferably hydroxypropyl methylcellulose, and an absorbent aid, preferably DL-malic acid.

The present invention provides an oral pharmaceutical formulation which is distinguishable from the above disclosed prior art compositions. The oral pharmaceutical formulation contains a core which has a diameter of about 18-20-mesh, which is significantly smaller than the size of the core described in the '015 patent. The core of the oral pharmaceutical formulation in the present invention is coated with a drug coating layer which contains an antifungal drug and a binder. The present formulation is further characterized by not containing an emulsion and an absorbent aid in the drug coating layer. Because of the increase in surface areas due to the use of smaller size (i.e., 18-20 mesh) cores, the present formulation demonstrates higher absorption and dissolution rates so as to enable the present inventors to use 50% less amount of the antifungal drug as used in Sporanox® by Janssen Pharmaceutica (Beerse, BE), the brand name drug maker, but still achieve the same therapeutic results. The present invention also has the advantage of providing the patients with flexible dosage forms due to its higher absorption and dissolution rates and superior bioavailability as compared to the commercially available azole antifungal drugs.

### SUMMARY OF THE INVENTION

The present invention provides an oral pharmaceutical formulation which contains (a) a core, preferably spherical or round shape, having a diameter of 18-20 mesh; and (b) a drug coating layer which contains an effective amount of an azole antifungal drug and a binder. The core and the antifungal drug has a ratio of about 1:0.2-0.6 by weight, preferably 1:0.25 to 0.55 by weight. This oral pharmaceutical formulation is further characterized for not containing an emulsion (such as polyoxypropylene-polyoxyethylene block copolymers, polyoxyethylene-sorbitan-fatty acid esters, sodium lauryl sulfate, or vitamin E polyethylene glycol succinate) and an absorbent aid (such as DL-malic acid, citric acid, ascorbic acid, or alginic acid).

The azole antifungal drug is preferably dissolved in organic solvents, including, but not limited to, methylene chloride, ethanol, or isopropanol. The preferred organic solvents are methylene chloride and ethanol at a ratio of about about 1.0 to 1.6-2.0 by volume.

Examples of the azole antifungal drugs are itraconazole, saperconazole, ketoconazole, and fluconazole. The most favorable drug is itraconazole.

The core of the oral pharmaceutical formulation is preferably made of a core material which is sucrose, lactose, starch, talc, or microcrystalline cellulose, or a mixture thereof.

The binder used in the drug coating layer of the oral pharmaceutical formulation is polyvinyl pyrrolidone (PVP), hydroxypropyl cellulose (HPC), hydroxypropyl methylcellulose (HPMC), or methylcellulose (MC), or a mixture thereof. It is preferable that the binder is about 25 to 52% by weight of the entire oral pharmaceutical formulation.

Optionally, the oral pharmaceutical formulation contains polyvinyl pyrrolidone (PVP K-30) as a plasticizer.

It is further optionally for the oral pharmaceutical formulation to contain a protective layer which is coated onto the drug coating layer. The protective layer is about 1 to 7% by weight of the oral pharmaceutical formulation. The preferred material for the protective layer is polyethylene glycol (PEG) at a molecular weight of 20,000.

The present invention also provides a method for making the oral pharmaceutical formulation which contains the following steps: (1) obtaining cores; (2) collecting the cores having a diameter of 18-20 mesh by passing the cores through a 18 inches sieve and 20 inches sieve respectively; (3) dissolving an azole antifungal drug and a binder in organic solvents to form a drug coating layer; and (4) spraying the drug coating layer onto the cores having a diameter of 18-20 mesh.

The cores are obtained by (1) dissolving polyvinyl pyrrolidone in isopropanol to produce a binder solution; and (2) spraying the binder solution onto sucrose to form the cores. Optionally, starch and talc can be added to the cores simultaneously when the binder solution is sprayed onto the sucrose.

Finally, the present invention provides a method for treating patients with fungal infection by orally administering the oral pharmaceutical formulation as shown above to the patients having fungal infection.

### BRIEF DESCRIPTION OF THE DRAWINGS

FIG. 1 shows the % dissolution of infra Example 1 (♦, solid diamond), and Sporanox® by Janssen pharmaceutica (Beerse BE) (▪, solid square) in 0.1 N HCl at various time points.

FIG. 2 shows the Mean (±S.E, standard error) plasma concentration profiles of itraconazole (ng/ml) in human after oral administrations of infra Examples 1 (▪, solid square), and Sporanox® by Janssen Pharmaceutica (Beerse, BE) (□, open square).

FIG. 3 shows the % dissolution of infra Example 2 (♦, solid diamond), Example 3 (▪, solid square), Example 4 (Δ, open triangle), and Example 5 (▴, solid triangle) in 0.1 N HCl at various time points.

FIG. 4 shows the Mean (±S.E, standard error) plasma concentration profiles of itraconazole (ng/ml) in human after oral administrations of infra Examples 5 (▪, solid square), and Sporanox® in capsule or tablet form from Janssen Pharmaceutica (Beerse, BE) (□, open square). The amount of itraconazole used in Example 5 was similar to that used in Sporanox®.

FIG. 5 shows the Mean (±S.E, standard error) plasma concentration profiles of itraconazole (ng/ml) in human after oral administrations of infra Examples 5 (▪, solid square), and Sporanox® in capsule or tablet form from Janssen Pharmaceutica (Beerse, BE) (□, open square), where the amount of itraconazole used in Example 5 was reduced in half (i.e., 50% dosage).

### DETAILED DESCRIPTION OF THE INVENTION

Azole antifungal or antibacterial agents, such as itraconazole, saperconazole, ketoconazole, or fluconazole, are extremely low in solubility and bioavailability. Therefore, these agents are difficult to administer orally. Although these agents are frequently prescribed for the treatment of fungal or bacterial infections, they are generally available in topical preparations or in oral formulations with limited bioavailability.

Due to limited bioavailability, it is generally recommended that these drugs be taken after meals to improve their bioavailability because these azole antimicrobial agents have a high binding rate with plasma proteins. For example, itraconazole has a binding rate with plasma proteins of 99.8%, which is also evidenced by the fact that the concentration of itraconazole in blood is about 60% of that in plasma.

The low solubility and bioavailability of itraconazole are also demonstrated by the fact that once itraconazole has been taken by the patients, it takes about 3-4 hours for the drug to reach a peak concentration in plasma. The plasma itraconazole has a half life of about 1 to 1.5 days. Also, itraconazole is primarily metabolized in the liver. However, only one of the metabolites of itraconazole, hydroxy-itraconazole, has been demonstrated in vitro to have antifungal activity. About 3-18% of the itraconazole in its original form is excreted in feces. About or less than 0.03% of the itraconazole in its original form is secreted in urine. About 35% of the itraconazole metabolites are secreted in urine within a week after the uptake of the drug.

The present invention is designed to improve the solubility and bioavailability of the itraconazole for use in oral administration. The pharmaceutical formulation is designed as shown in the following diagram: 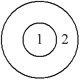
Where 1 represents a granular core, and 2 represents a drug coating layer. The core is about 18-20-mesh in diameter. The term “mesh” is used in accordance with the pharmaceutical industrial standards to mean the size of the sugar spheres based on the number of sieve openings per surface unit. According to the standards used in the pharmaceutical industry, cores are measured and labeled based on the size sieve they fall through. For the 18-20 mesh cores, the cores should fall through a 18 mesh sieve but stays on top of the 20 mesh sieve. The 18-20 mesh cores generally have diameters in the range of 840-1000 μm. The drug coating layer contains the azole antifungal drug (such as itraconazole) and a binder. The drug coating layer is further characterized as containing no emulsion agent (such as polyoxypropylene-polyoxyethylene block copolymers, polyoxyethylene-sorbitan-fatty acid esters, sodium lauryl sulfate, or vitamin E polyethylene glycol succinate). In addition, the drug coating layer does not contain an absorbent aid (such as DL-malic acid, citric acid, ascorbic acid, and alginic acid).

Optionally, a protective layer can be added to cover the drug coating layer as shown in the following scheme:

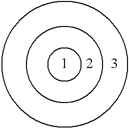

The protective layer is designated as 3 as shown in the above scheme. The preferred material to be used in the protective layer is polyethylene glycol, having a molecular weight ranged from 6000 to 20,000.

A general description regarding how the pharmaceutical formulation of the present invention is made is provided as follows:

(A) The Core:

The cores of the pharmaceutical formulation are made of rounded or spherical edible particles. Materials suitable for use as cores include, but are not limited to, sucrose, lactose, starch, talc, and microcrystalline cellulose, which can be utilized solely or as a mixture at any combination and given ratios. The cores are obtained either by direct purchase from bulk drug manufacturers or in-house preparation. There are generally three kinds of neutral, edible cores which are commercially available. They are (a) 100% pure sucrose cores; (b) combined sucrose and starch cores; and (c) microcrystalline cellulose cores.

The cores made by in-house preparation generally follow the manufacturing process as follows:

1. adding 40 g of polyvinylpyrrolidone to 300 mL of isopropyl alcohol and 200 mL of distilled water to form a binder mixture. Stir the binder mixture until all are dissolved;

2. weighing 400 g of sucrose;

3. weighing 800 g of starch and 900 g of talc and mix until homogeneous;

4. spraying the binder mixture (1) onto the surface of sucrose (2), while at the same time mixing (3) with the sprayed sucrose particles to form wet granular cores;

5. drying the wet cores to form the dried cores; and

6. passing the dried cores through an 18-inch sieve once and a 20-inch sieve once. Taking the cores that falls within the 18-20 mesh (1.0 mm to 0.84 mm) to be used for the preparation of the present pharmaceutical formulation.

The in-house preparation of the cores can be carried on in a fluidized-bed centrifuge granulator (Glatt).

(B) Drug Coating Layer

The drug coating layer contains an azole antifungal drug (such as itraconazole) as the active ingredient, and a binder. The azole antifungal drug is dissolved in organic solvents.

1. The active ingredient includes, but is not limited to, itraconazole, saperconazole, ketoconazole, or fluconazole.

2. The binder includes, but is not limited to, polyvinylpyrrolidone, hydroxypropylcellulose, hydroxypropylmethylcellulose, or methylcellulose, or a mixture thereof.

3. The organic solvents to be used for dissolving the active ingredient include, but are not limited to, methylene chloride, ethanol, and isopropyl alcohol. The preferred organic solvents are a mixture of methylene chloride and ethanol in a ratio of about 1.0:1.6-2.0 by volume.

The process for making the drug coating layer is shown as follows:

1. Mixing the binder (such as hydroxypropylmethylcellulose) and the active ingredient (such as itraconazole) in a stainless steel container and adding ethanol to the mixture of the binder and the active ingredient. Mixing all the ingredients until homogeneous. Adding methylene chloride and mixing until all are dissolved to form a drug coating solution.

2. Spraying the aforementioned drug coating solution onto the 18-20-mesh granular cores for coating to form the wet granules, followed by drying the wet granules to form the pharmaceutical formulation of the present invention.

(C) Protective Layer

Optionally, a protective layer, preferably containing polyethylene glycol (PEG) in the molecular weight between 6,000 to 20,000, can be sprayed onto the dried drug coating layer to serve a seal-coating spray for the pharmaceutical formulation. PEG is preferably to be added to a beaker. A suitable quantity of distilled water is then added to the beaker containing the PEG and the water and PEG are stirred until the PEG is dissolved to form a sealing solution.

This sealing solution is then sprayed onto the dried drug coating layer, followed by drying.

The present invention significantly improves the slow absorption problem associated with itraconazole-containing hard capsules by the body by directly coating the drug coating solution containing the main ingredient and a binder on the surface of the 18-20-mesh granular cores (1.0 mm to 0.84 mm in diameter) to increase the contact surface of itraconazole in the body. The current commercially available itraconazole pellets are using 25-30 mesh cores (i.e., having a diameter of 600 to about 700 μm). A larger size core increases the contact surface of the drug in the body so as to provide stable dissolution and improve the therapeutic effects. As a result, the pharmaceutical formulation according to the present manufacturing method demonstrates superior dissolution, which is conformed to the standards set forth in the 22^{nd} Edition of U.S. Pharmacopoeia. Additionally, due to the relatively simple operation process without going through many manufacturing steps, the pharmaceutical formulation of the present invention has the advantages of improving the production rate, reducing the manufacturing time, and not being relied upon a particular machine for production. Either the fluidized-bed centrifuge granulator (Glatt) or the fluidized-bed spraying granulator (Huttlin) can be employed in the production of the present pharmaceutical formulation.

The following examples describe the pharmaceutical formulations using itraconazole as an example of the azole antimicrobial drug, and the process of making the formulations. These examples are for illustrative purposes. They should not be viewed as limitations of the scope of the present invention. Reasonable variations, such as those that occur to a reasonable artisan, can be made herein without departing from the scope of the present invention.

### EXAMPLE 1

(A) Materials and Method for Preparation of the Cores:

The cores were prepared using the following ingredients:

| Ingredients | Amount | |
|---|---|---|
| Polyvinyl Pyrrolidone (PVP K-30) | 40 | g |
| Isopropyl Alcohol | 300 | ml |
| Purified Water | 200 | ml |
| Sucrose | 400 | g |
| Starch | 800 | g |
| Talc | 900 | g |

The cores were produced by a three-step process. The first step included dissolving 40 g of PVP K-30 in 300 ml of isopropyl alcohol with stirring and then mixing with 200 ml of distilled water, which produced a binder solution. The second step included mixing 800 g of starch and 900 g of talc together. The final step included putting sucrose into a fluidized bed granulator (such as Glatt or Huttlin) and spraying the PVP K-30 binder solution produced in the first step onto the sucrose, while at the same time adding the starch-talc mixture to the sucrose, to form the cores. The cores were further dried under warm air.

The dried cores were then passed through an 18-inch sieve once and a 20-inch sieve once. the cores having the size within 18-20 mesh were retained for use in the manufacturing of the granules of the present invention.

(B) Materials and Method for Preparation of the Drug Coating Layer

The drug coating layer was prepared using the following ingredients:

| INGREDIENTS | AMOUNT | |
|---|---|---|
| Itraconazole | 600 | g |
| Hydroxypropyl Methylcellulose (HPMC) | 1026 | g |
| Methylene Chloride | 6900 | ml |
| Ethanol | 12600 | ml |

The drug coating layer was prepared by mixing 1026 g of HPMC and 600 g of itraconazole, followed by adding 12600 ml of ethanol to the mixture until all of the ingredients were thoroughly mixed. Then, 6900 ml of methylene chloride were added to the mixture until all of the ingredients were completely dissolved to form the drug coating layer.

(C) Method for Making the Pharmaceutical Formulation:

The drug coating layer-coated granules were prepared by placing the 1074 g of the 18-20 mesh cores as described in (A) into a fluidized-bed centrifuge granulator (Glatt). The drug coating layer as described in (B) was sprayed, as a mist-like solution, onto the cores while the Glatt was in operation to form wet granules, which were further dried to form the drug coating layer-coated granules of the present pharmaceutical formulation.

### EXAMPLE 2

The cores and the drug coating layer of the pharmaceutical formulation of Example 2 were prepared according to the procedures described in Example 1 except that the quantity of the cores used in Example 2 was different from that in Example. The pharmaceutical formulation of Example 2 contained the following ingredients:

| INGREDIENT | AMOUNT | |
|---|---|---|
| 1. The Cores: | 1155 | g |
| 2. The Drug Coating Layer: | | |
| Itraconazole | 600 | g |
| Hydroxypropyl Methylcellulose (HPMC) | 945 | g |
| Methylene Chloride | 6900 | ml |
| Ethanol | 12600 | ml |

### EXAMPLE 3

The cores and the drug coating layer of the pharmaceutical formulation of Example 3 were the same as those described in Examples 1-2, except that the pharmaceutical formulation of Example 3 contained a protective layer, which was used as a seal coating for the drug coating layer. The pharmaceutical formulation of Example 3 contained the following ingredients:

| INGREDIENT | AMOUNT | |
|---|---|---|
| 1. The Cores: | 1155 | g |
| 2. The Drug Coating Layer: | | |
| Itraconazole | 600 | g |
| Hydroxypropyl Methylcellulose (HPMC) | 945 | g |
| Methylene Chloride | 6900 | ml |
| Ethanol | 12600 | ml |
| 3. The Protection Layer: | | |
| Polyethylene glycol (PEG) 20000 | 27 | g |
| Distilled Water | 270 | ml |

The protective layer was prepared by adding distilled water to PEG 20,000, followed by stirring until PEG 20,000 was completely dissolved.

After the drug coating layer-coated granules were made and dried in the Glatt, the protective layer was sprayed onto the drug coating layer-coated granules while the Glatt was still centrifuging to coat the protective layer onto the granules. The protective layer-coated granules were then dried to form the pharmaceutical formulation of the present invention.

### EXAMPLE 4

The cores and the drug coating layer of the pharmaceutical formulation of Example 4 were the same as described in Examples 1-3. Additionally, Example 4 contained a protective layer which was prepared according to the same procedure as described in Example 3, except for the quantities of the protective layer ingredients. The pharmaceutical formulation of Example 4 was prepared using the following ingredients:

| INGREDIENT | AMOUNT | |
|---|---|---|
| 1. The Cores: | 1155 | g |
| 2. The Drug Coating Layer: | | |
| Itraconazole | 600 | g |
| Hydroxypropyl Methylcellulose (HPMC) | 945 | g |
| Methylene Chloride | 6900 | ml |
| Ethanol | 12600 | ml |
| 3. The Protection Layer: | | |
| Polyethylene glycol (PEG) 20000 | 54 | g |
| Distilled Water | 360 | ml |

### EXAMPLE 5

The cores and the drug coating layer of the pharmaceutical formulation of Example 5 were the same as described in Examples 1-4. Additionally, Example 5 contained a protective layer which was prepared according to the same procedure as described in Examples 3-4, except for the quantities of the protective layer ingredients. The pharmaceutical formulation of Example 5 was prepared using the following ingredients:

| INGREDIENT | AMOUNT | |
|---|---|---|
| 1. The Cores: | 1155 | g |
| 2. The Drug Coating Layer: | | |
| Itraconazole | 600 | g |
| Hydroxypropyl Methylcellulose (HPMC) | 945 | g |
| Methylene Chloride | 6900 | ml |
| Ethanol | 12600 | ml |

### Dissolution Test and Human Plasma Concentration Profile of Example 1

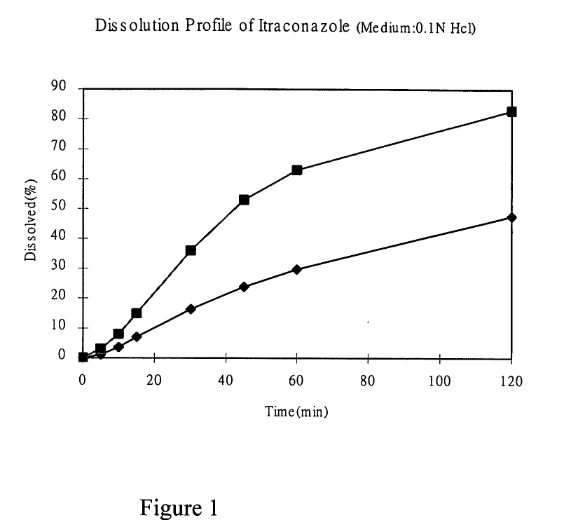

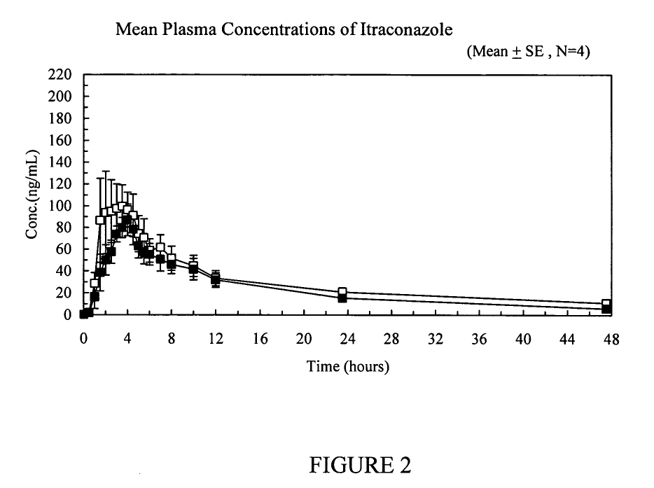

The dissolution rates (% dissolution) and human plasma concentrations of the itraconazole-containing pharmaceutical formulation described in Example 1, as compared to the Sporanox® capsules, were determined according to the method described in the 22^{nd} Edition of U.S. Pharmacopoeia. The results of these tests are shown in FIGS. 1 and 2, respectively.

The results of the % dissolution of itraconazole at 0 to 120 minutes, as shown in FIG. 1, demonstrated that % dissolution of Example 1 was lower than that of the commercially available itraconazole capsule (Sporanox®). These results could be attributed to the larger size of the cores (18-20 mesh) as used in Example 1, which provided less surface areas, than the smaller-size cores (25-30 mesh) used in the commercially available itraconazole capsule, which provided greater surface areas.

The results of the time course of the blood concentrations of itraconazole of Example 1 in human, as compared with those in the commercially available itraconazole capsule (Sporanox®), are shown in FIG. 2. The results showed that even though the % dissolution of Example 1 was lower than that of Sporanox®, the rate of absorption of itraconazole by the body, as reflected by the blood itraconazole concentrations, were not significantly different. This indicated that the absorption of Example 1 by the body is superior to that of Sporanox®, despite the lower % dissolution of the pharmaceutical formulation in Example 1.

### Dissolution Test and Human Plasma Concentration Profile of Examples 2-5

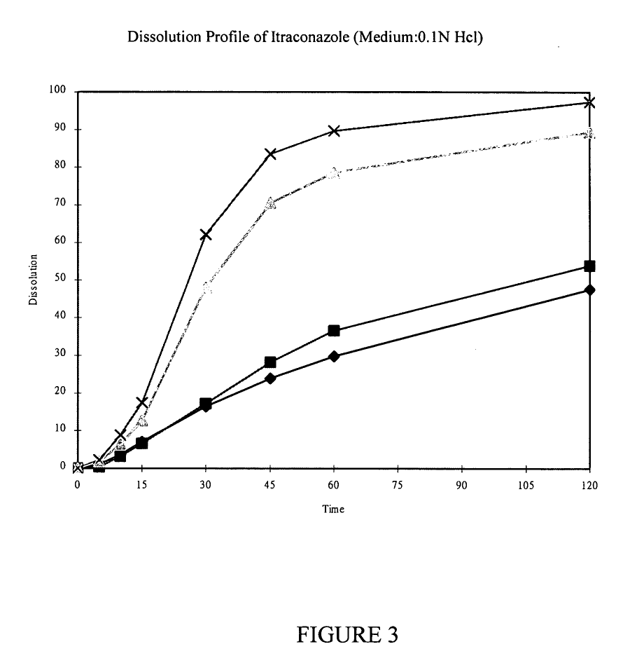

FIG. 3 showed the results of the % dissolution studies among Examples 2-5. The cores and the drug coating layer of Examples 2-5 were identical. The only differences among these Examples were the contents of PEG 20,000 used in the protective layer, with Example 2 containing the least amount of PEG 20,000 and Example 5 containing the highest amount.

The results of FIG. 3 showed that % dissolution increased proportionally to the amount of PEG 20,000 used in the protective layer, the lower the PEG 20,000, the lower the % dissolution.

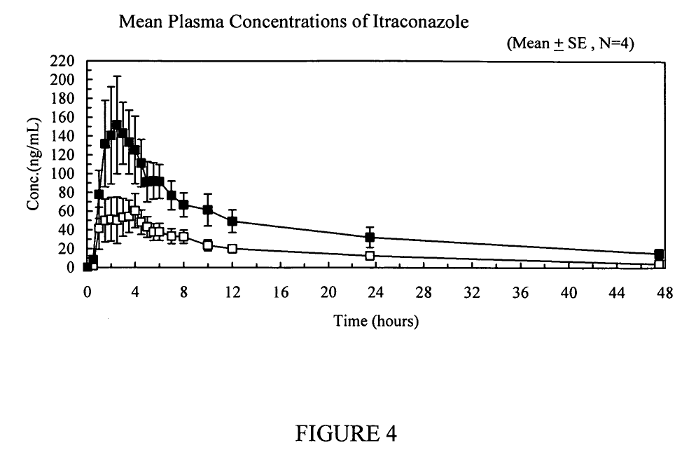

FIG. 4 showed the time course of the blood concentrations of itraconazole of Example 5 in human, as compared with those in the commercially available itraconazole capsule (Sporanox®). Clearly the results demonstrated that the rate of absorption of itraconazole in Example 5 by the human body is superior to that in Sporanox®.

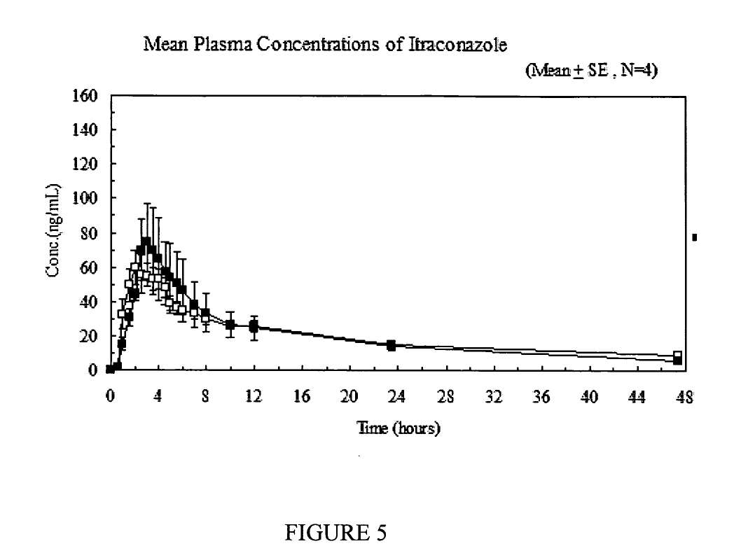

For the purpose of establishing bioequivalency, the quantity of itraconazole in Example 5 was reduced in half (i.e., reducing to 50%) and the time course of the plasma concentrations of the 50% reduced Example 5, as compared with those in the commercially available itraconazole capsule (Sporanox®), was studied. As shown in FIG. 5, the differences in the mean plasma concentrations of itraconazole in the 50% reduced Example 5 and the commercially available itraconazole capsule (Sporanox®) were insignificant. This indicated that the rate of absorption in Example 5 was about twice higher than that of Sporanox®.

While the invention has been described by way of examples and in terms of the preferred embodiments, it is to be understood that the invention is not limited to the disclosed embodiments. On the contrary, it is intended to cover various modifications as would be apparent to those skilled in the art. Therefore, the scope of the appended claims should be accorded the broadest interpretation so as to encompass all such modifications.

We claim:

1. An oral pharmaceutical formulation comprising:
a core having a diameter of 18-20 mesh; and
a drug coating layer which comprises an effective amount of an azole antifungal drug and a binder;
wherein said core and said antifungal drug has a ratio of about 1:0.2-0.6 by weight;
wherein said oral pharmaceutical formulation does not contain an emulsion; and
wherein said oral pharmaceutical formulation does not contain an absorbent aid.

2. The oral pharmaceutical formulation according to claim 1, wherein said azole antifungal drug is dissolved in organic solvents.

3. The oral pharmaceutical formulation according to claim 2, wherein said organic solvents are selected from the group consisting of methylene chloride, ethanol, and isopropanol.

4. The oral pharmaceutical formulation according to claim 2, wherein said organic solvents are a mixture of methylene chloride and ethanol at a ratio of about 1.0 to 1.6-2.0 by volume.

5. The oral pharmaceutical formulation according to claim 1, wherein said azole antifungal drug is itraconazole.

6. The oral pharmaceutical formulation according to claim 1, wherein said azole antifungal drug is saperconazole, ketoconazole, or fluconazole.

7. The oral pharmaceutical formulation according to claim 1, wherein said core comprises a core material which is at least one selected from the group consisting of sucrose, lactose, starch, talc, and microcrystalline cellulose.

8. The oral pharmaceutical formulation according to claim 1, wherein said binder is one selected from the group consisting of polyvinyl pyrrolidone (PVP), hydroxypropyl cellulose (HPC), hydroxypropyl methylcellulose (HPMC), and methylcellulose (MC).

9. The oral pharmaceutical formulation according to claim 1, wherein said binder is about 25 to 52% by weight of said oral pharmaceutical formulation.

10. The oral pharmaceutical formulation according to claim 1, wherein said emulsion is at least one selected from the group consisting of polyoxypropylene-polyoxyethylene block copolymers, polyoxyethylene-sorbitan-fatty acid esters, sodium lauryl sulfate, or vitamin E polyethylene glycol succinate.

11. The oral pharmaceutical formulation according to claim 1, wherein said absorbent aid is at least one selected from the group consisting of DL-malic acid, citric acid, ascorbic acid, and alginic acid.

12. The oral pharmaceutical formulation according to claim 1, further comprising polyvinyl pyrrolidone (PVP K-30) as a plasticizer.

13. The oral pharmaceutical formulation according to claim 1, further comprising a protective layer coated onto said drug coating layer.

14. The oral pharmaceutical formulation according to claim 13, wherein said protective layer is about 1 to 7% by weight of the oral pharmaceutical formulation.

15. The oral pharmaceutical formulation according to claim 1, wherein said protective layer comprises polyethylene glycol (PEG) at a molecular weight of 20,000.

16. The oral pharmaceutical formulation according to claim 13, wherein said azole antifungal drug of said oral pharmaceutical formulation is itraconazole, and wherein said oral pharmaceutical formulation has a rate of absorption of itraconazole in human body about twice of that of Sporanox®.

17. A method for making the oral pharmaceutical formulation according to claim 1, comprising:
obtaining said core;
collecting said core having a diameter of 18-20 mesh by passing said core through a 18 inches sieve and 20 inches sieve respectively;
dissolving said azole antifungal drug and said binder in said organic solvents to form a drug coating layer; and
spraying said drug coating layer onto said core having a diameter of 18-20 mesh.

18. The method according to claim 17, wherein said core is obtained by
dissolving polyvinyl pyrrolidone in isopropanol to produce a binder solution; and
spraying said binder solution onto sucrose to form said core.

19. The method according to claim 18, further comprising:
adding starch and talc to said core simultaneously when said binder solution is sprayed onto said sucrose.

20. A method of treating patients with fungal infection comprising orally administering said oral pharmaceutical formulation according to claim 1 to said patients.
